(19) United States
(12) Patent Application Publication
(10) Pub. No.: US 2024/0000441 A1
(43) Pub. Date: Jan. 4, 2024

(54) Spatially-Selective Sampling of Gut Microbiome

(71) Applicant: Trustees of Tufts College, Medford, MA (US)

(72) Inventors: Sameer Sonkusale, Lincoln, MA (US); Hojatollah Rezaei Nejad, Newton, MA (US); Aydin Sadeqi, Medford, MA (US)

(21) Appl. No.: 18/037,430

(22) PCT Filed: Nov. 18, 2021
(86) PCT No.: PCT/US2021/059826

§ 371 Date: May 17, 2023

Related U.S. Application Data
(60) Provisional application No. 63/115,355, filed on Nov. 18, 2020.

(51) Int. Cl. A61B 10/00; A61B 5/06; A61B 5/103

(52) U.S. Cl. CPC A61B 10/0045; A61B 5/062; A61B 5/1032; A61B 2562/162

(57) ABSTRACT

An orally-administered gut sampler traverses the gut and obtains a sample of a bacterial population that is present at a selected location within the gut. The sampler has a body that extends from a proximal end to a distal end along a longitudinal axis thereof. The body includes an inlet at the proximal end and a sample chamber distal to the inlet. A sample of bacteria begins to pass into the inlet and into the sample chamber when the sampler arrives at the selected location.

### RELATED APPLICATIONS

This application claims the benefit of the Nov. 18, 2020 priority date of U.S. provisional application 63/115,355, the contents of which are herein incorporated by reference.

### FIELD OF INVENTION

The invention pertains to bacterial demographics, and in particular, to identifying the spatial distribution of bacterial species within a region that is not easily accessible.

### BACKGROUND

The microbiome that is found within the gastrointestinal tract, i.e., the “gut microbiome,” has profound effects on the development and maintenance of the immune system in both animal models and in humans. A growing body of evidence has implicated the human gut microbiome in a range of disorders, including obesity, inflammatory-bowel diseases, cancer, and cardiovascular disease.

The gut microbiome has a significant population of bacteria, typically on the order of 100 trillion individual cells. Most of these cells belong to a thousand or so bacterial species. Studies examining this bacterial population have shown wide variations in which species are present between individuals.

The gastrointestinal tract, particularly those portions rich in bacteria, is difficult to access. One cannot simply swab a particular portion of the gastrointestinal tract to obtain a sample of the bacterial population at that portion. Instead, the usual procedure is to analyze fecal matter.

A difficulty that arises with the inspection of fecal matter is that it inevitably traverses the entire gastrointestinal tract, picking up various species of bacteria along the way. As a result, although fecal matter provides information on what species are present, it does not provide information on where those species were found.

To gain new insights into the role of gut microbiome, it is useful to sample different locations in the gastrointestinal tract to obtain a spatial distribution profile. Such studies are currently not possible with the fecal matter analysis.

### SUMMARY

This invention relates to an orally-administered pill that travels through the gut and obtains samples of the microbiome in such a way that the location from which the sample was taken can be identified. As used herein, the “gut” includes both the large intestine and the small intestine.

In one aspect, the invention features an orally-administered gut sampler that traverses the gut and that obtains a sample of a bacterial population that is present at a selected location within the gut. The sampler has a body that extends from a proximal end to a distal end along a longitudinal axis thereof. The body includes one or more inlets and a sample chamber. A sample of bacteria begins to pass into one or more of the inlets and into the sample chamber when the sampler arrives at the selected location. In some embodiments, the inlet is at the proximal end and the sample chamber is distal to the inlet.

In some embodiments, the further includes a channel that extends from the inlet to the sample chamber. Among these are embodiments in which the sample chamber includes a bag having a volume that has been reduced as a result of having been folded within the body.

In other embodiments, the gut sampler further includes a channel and a valve. The channel extends between the inlet and the sample chamber. The valve is biased to block the channel. In such embodiments, the sample chamber is configured to expand in volume, thereby causing a pressure differential across the valve. This pressure differential is sufficient to open the valve. It also diminishes over time. The valve thus closes when the pressure differential is no longer sufficient to overcome the bias.

Still other embodiments include a coating around the body. The coating is configured to begin to dissolve in response to exposure to fluid that is found at the selected location. Among these are coatings that dissolve in acidic environments and coatings that dissolve in alkaline environments.

In some embodiments, the sample chamber is distal to an osmotic membrane. In these embodiments, a channel extends from the inlet to the osmotic membrane. Among these are embodiments the inlet is one of plural inlets, all of which open into a stilling chamber. Such embodiments include plural channels, each of which connects the stilling chamber to the osmotic membrane.

Embodiments further include those in which a first piece of material forms a first plug in a channel that connects to the inlet and a second piece of material is located so as to be transformable into a second plug that blocks the channel.

Still other embodiments features a valve and electric heaters that are disposed to heat first and second portions of the valve.

Also among the embodiments of the gut sampler are those that have a magnet and those that include a fluorescent marker.

Still other embodiments of the gut sampler further include a valve and a controller configured to control actuation of the valve.

Additional embodiments includes a voltage source, switches, and resistances. The switches transition into respective closed states that permit current driven by the voltage source to flow through respective ones of the resistances.

Embodiments further include those in which the gut sampler includes a screw and a motor coupled to the screw to cause rotation of the screw. Among these are embodiments in which the screw extends through a tube that extends from the inlet to a point proximal to the motor where the tube is in fluid communication with the sample chamber. In such embodiments, the screw, when being rotated by the motor, draws sample through the tube and into the sample chamber.

Other embodiments include a panel that rotates about the longitudinal axis between first and second positions. In the first position, the panel opens the inlet. In the second position, the panel closes the inlet.

Also among the embodiments that include a panel are those in which the panel translates along the longitudinal axis between first and second positions that are offset from each other along the longitudinal axis. In such embodiments, the panel, when in its first position, closes the inlet and opens the inlet when it is in its second position.

Still other embodiments include those that have a shape-memory material and a heater that heats the shape-memory material to cause a transition between first and second states of the shape-memory material. The transition between the first and second states of the shape-memory material causes the inlet to transition between being closed and being opened.

Also among the embodiments are those in which the gut sampler does not rely on an external signal to operate but instead measures a property of its environment. Among these are embodiments in which the gut sampler includes a valve actuator that is actuated in response to a measurement of a property of an environment in which the gut sampler is located. In some embodiments, electrodes that are exposed to an environment in which the gut sampler is located carry out the measurement and a controller that receives signals from the electrodes and opens and closes the inlet in response to those signals. In other embodiments, the measurement is carried out optically. In such embodiments, the gut sampler comprises a photodetector, a light source, a dye source, and a controller that receives a signal from the photodetector. The light source is configured to illuminate a dye released by the dye source and the photodetector is disposed to view the dye and to provide a signal indicative of the dye's state.

### BRIEF DESCRIPTION OF THE DRAWINGS

FIG. 1 is an isometric view of a sampler that draws a sample into a foldable bag;

FIG. 2 shows a cross section of the sampler shown in FIG. 1;

FIG. 3 shows the sampler of FIG. 1 in the process of sampling as the foldable bag unfolds;

FIG. 5. shows a sampler that relies on osmotic pressure to collect a sample;

FIG. 6 shows the valve of the sampler in FIG. 5 prior to sampling;

FIG. 7 shows the valve of the sampler of FIG. 5 during sampling;

FIG. 8 shows the valve of the sampler of FIG. 5 after sampling;

FIG. 9 shows circuitry for causing the valve of the sampler in FIG. 5 to transition through the states shown in FIGS. 6-8;

FIG. 10 shows a variant of the sampler shown in FIG. 5 but with multiple channels and inlets;

FIG. 11 shows a sampler that samples using a motorized screw;

FIG. 12 shows a sampler similar to that in FIG. 11 but with a smaller motorized screw;

FIG. 13 is an exploded view of the sampler shown in FIG. 12;

FIG. 14 shows a sampler having a valve that has been closed by rotating a panel;

FIG. 15 shows the valve of FIG. 14 with the panel having been rotated to open the valve.

FIG. 16 shows a top view of the closed valve shown in FIG. 14;

FIG. 17 shows a top view of the open valve shown in FIG. 15;

FIG. 18 shows a sampler having a valve that has been closed by a shape-memory alloy that, in its relaxed state, pulls a panel in a distal direction;

FIG. 19 shows the valve of FIG. 19 after the shape-memory alloy has been heated into an expanded state in which it lifts the panel in a proximal direction;

FIG. 20 shows a sampler having a valve that has been closed as a result of having heated a shape-memory allow into an expanded state;

FIG. 21 shows the valve of FIG. 20 having been opened as a result of the shape-memory alloy having reverted to its relaxed state;

FIG. 22 shows a sampler having an autonomous sensor that relies on three electrodes;

FIG. 23 shows a sampler having an autonomous sensor that relies on biomarker-sensitive dyes; and

FIG. 24 shows optical components for inspecting the state of the dyes shown in FIG. 23.

### DETAILED DESCRIPTION

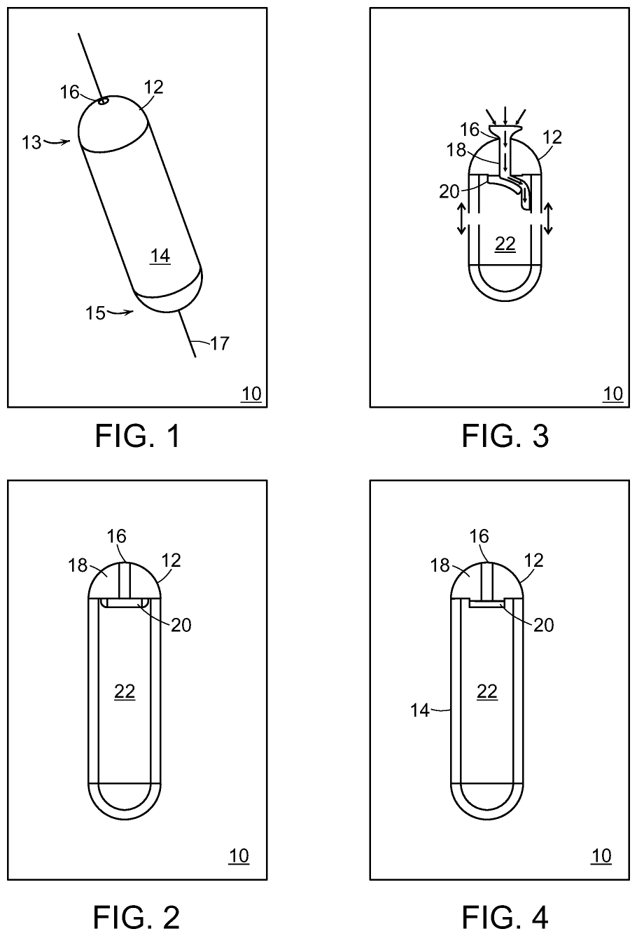

FIGS. 1 and 2 show a sampler 10 having a proximal cap 12 at a proximal end 13 thereof. The proximal cap 12 connects to a body 14 that extends from the proximal cap 12 towards a distal end 15 of the sampler 10 along a longitudinal axis 17. An inlet 16 on the proximal cap 12 opens into a proximal end of a channel 18 that likewise extends towards the cap's distal end 15. The body 14 and proximal cap 12 define a capsule that is readily swallowed by a patient.

A valve 20 along a distal end of the channel 18 controls flow through the channel 18. In the illustrated embodiments, a bias force urges this valve 20 to remain closed, thus preventing flow through the channel 18. The valve's biasing force arises from the valve's elasticity.

The sampler 10 comprises a sample chamber 22 formed by a bag. This bag is initially folded. As a result, the sample chamber 22 has a minimal volume.

The body 14 comprises a material that dissolves while the sampler 10 is within a patient's gastrointestinal tract. As the body 14 dissolves, the bag unfolds, thus expanding the volume of the sample chamber 22.

As the body 14 dissolves, the bag unfolds, thus expanding the sample chamber 22, as shown in FIG. 3. Because the bag was initially folded, there is essentially no gas in the sampling chamber 22. The resulting pressure differential between the sampling chamber 22 and the space outside the sampler 10 causes a suction force that overcomes the valve's bias force, thus opening the valve 20 and drawing sample through the inlet 16. The sample flows distally through the channel 18 and into the sample chamber 22.

As the sample chamber 22 fills, as shown in FIG. 4, the pressure equalizes and therefore the suction decreases. Eventually, it is no longer enough to overcome the valve's biasing force, at which point the valve 20 closes. This locks the sample into the sample chamber 22 and preventing additional sample from contaminating it. As a result, the sample is guaranteed to be from a particular spatial portion of the gastrointestinal tract.

The remaining portions of the sampler 10, which include the sample chamber 22 and the proximal cap 12, continue to traverse the gastrointestinal tract and are eventually recovered following defecation. To promote recoverability, it is useful for the proximal cap 12 to be made visually conspicuous, for example by incorporating a fluorescent material or by dyeing it with a color that is sufficiently distinct from that of fecal matter.

In a typical embodiment, the channel 18 is on the order of five millimeters long and has a diameter that is small enough so that surface effects dominate flow of fluid through the channel. Such a channel is thus regarded as a microfluidic channel.

A suitable material from which to make the body 14 is gelatin. By controlling the thickness of the gelatin, it is possible to control, to some extent, where the body 14 will dissolve sufficiently to release the sample chamber 22. In some embodiments, the body 14 is configured to release the sample chamber 22 in the small intestine. In others, it is configured to release the sample chamber 22 in the large intestine.

The concentrations of electron donors and electron acceptors varies as a function of location along the gastrointestinal tract. As such, it is possible to control the location from which a sample is taken by exercising control over the material from which the body 14 is made.

In some embodiments, an enteric coating over the sampler 10 dissolves in response to exposure to the environment in which sampling is to take place. For example, if a sample from the stomach is sought, the enteric coating is one that dissolves upon exposure to a high concentration of electron acceptors, such as is found in the stomach. In the alternative, if a sample is sought from large intestine or the small intestine, the enteric coating is one that resists being dissolved in the stomach and only dissolves upon exposure to the environment of the relevant intestine.

By suitable selection of the material from which the enteric coating is made, it is possible to target the region to be sampled with considerable specificity. As such, it is possible to cause the enteric coating to dissolve within the duodenum, jejunum, or ileum of the small intestine or within the proximal or distal colon.

In operation, the sampler 10 is orally administered and makes its way through the gastrointestinal tract through peristalsis. At some point in its journey through the canal, the sampler 10 opens up to receive a sample and promptly closes again. The sampler 10 is then recovered from the feces upon eventual excretion carrying with it an uncontaminated sample from a particular portion of the gastrointestinal tract.

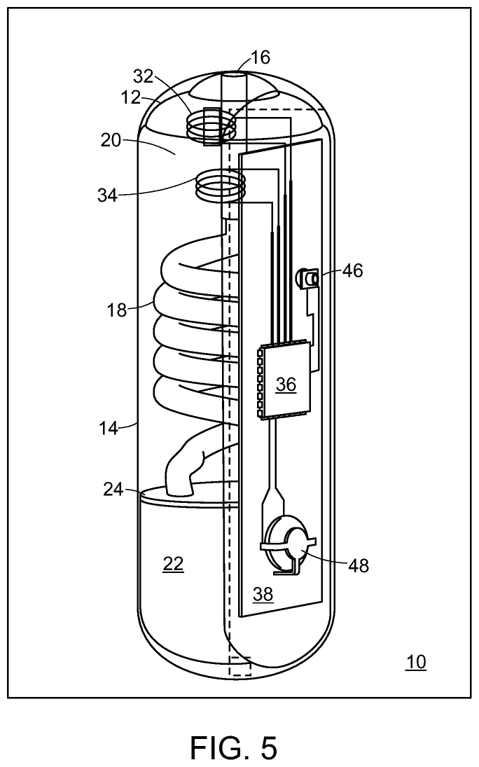

In another embodiment, shown in FIG. 5, the sampler 10 includes an osmotic membrane 24 that defines the sample chamber 22 at an end of the sampler's body 14. In this embodiment, the channel 18 traverses a helical path between the inlet 16 and the osmotic membrane 24.

In this embodiment, the valve 20 takes the form of a first plug 28 and a second plug 30. A valve actuator for opening and closing the valve 20 comprises a first heater 32 that is adjacent to the first plug 28 and a second heater 34 that is adjacent to the second plug 30. A controller 36 controls operation of the first heater 32 and the second heater 34.

The first plug 28 and the second plug 30 are made of a material that transitions between solid and liquid phase at a temperature that is achievable by the first heater 32 and the second heater 34. A suitable material is a wax, such as paraffin wax. Other suitable materials are those with a melting point between 35° C. and 70° C., and in particular, those with a melting point of between 44° C. and 48° C.

The first and second heaters 32, 34 take the form of coiled wires that have surround the channel at first and second locations corresponding to the first and second plugs. Current through these wires generates heat for melting the first and second plugs 28, 30.

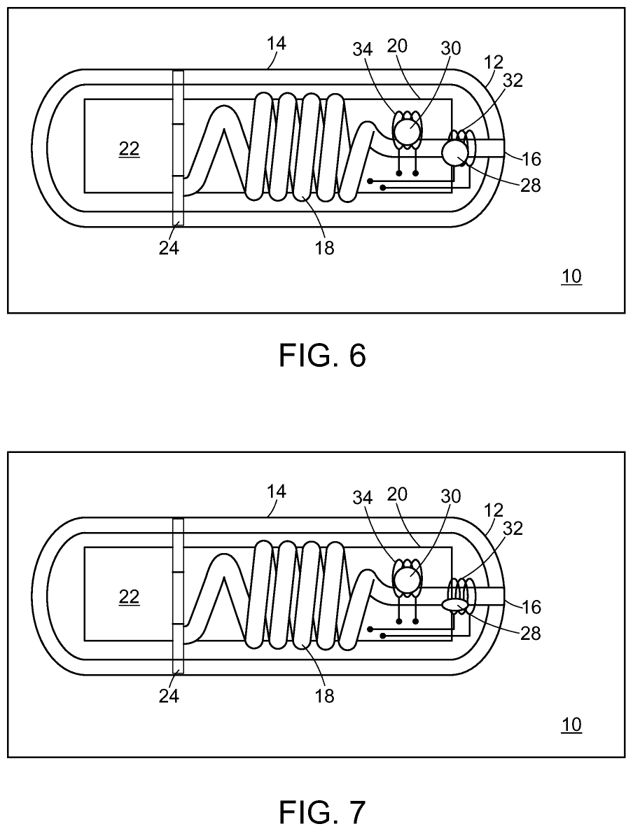

As shown in FIG. 6, the sampler 10 is manufactured in a first closed-state in which the first plug 28 is disposed in the channel 18 and the second plug 30 is outside the channel 18. As a result, the first plug 28 blocks the channel and the second plug 30 has no effect on the channel 18.

The controller 36 causes the valve 20 to transition from the first closed-state to an open state, which is shown in FIG. 7. It does so by causing the first heater 32 to melt the plug 28. This opens up the channel 18 and allows sample to flow towards the osmotic membrane 24.

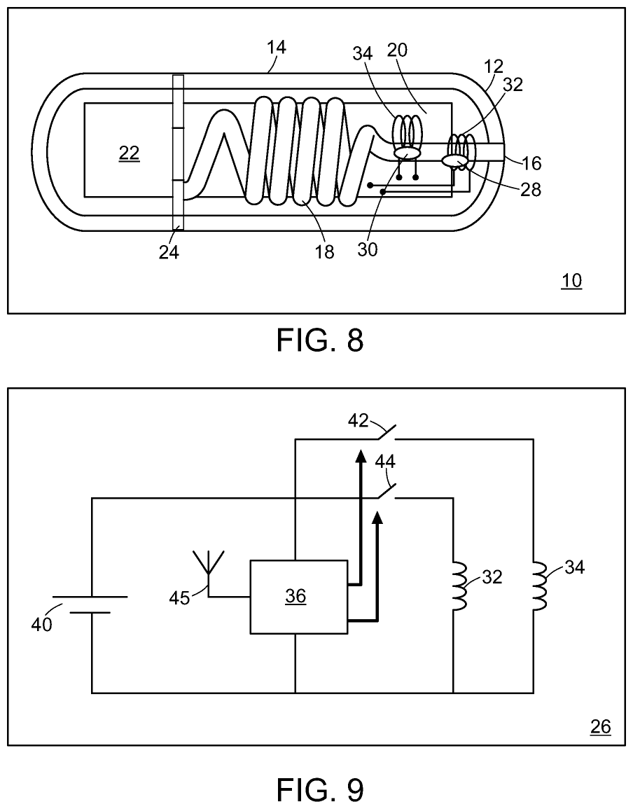

Upon lapse of a suitable sampling interval, the controller 36 causes the valve 20 to transition into a second closed-state, which is shown in FIG. 8. It does so by causing the second heater 34 to melt the second plug 30. The second plug 30, as a result of having been melted, flows into the channel 18, where it resolidifies. As a result, the second plug 30 blocks the channel 18 in much the same way that the first plug 28 originally blocked the channel 18.

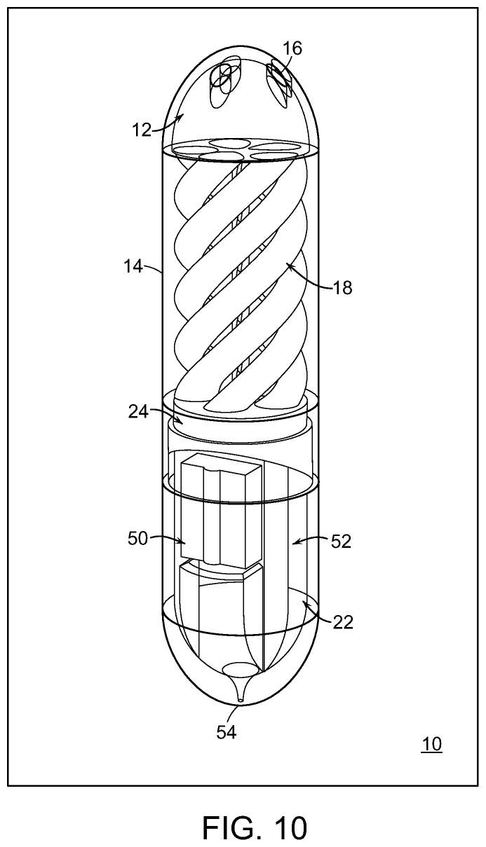

Referring back to FIG. 5, a flexible printed circuit board 38 within the sampler 10 carries the controller 36 together with circuitry 26 shown in FIG. 9. The illustrated circuitry features a voltage source 40, and first and second switches 42, 44 that the controller 36 opens or closes to provide current to the first and second heaters 32, 34, respectively.

An antenna 45 connected to an antenna port 46 receives an external signal that causes the controller 36 to open and close the relevant first and second switches 42, 44 at times when a sample is to be taken. A suitable frequency is one at which human tissue permits adequate penetration, for example at or near 433 megahertz. A suitable controller 86 incorporates a radio-frequency transceiver, such as the ATA8510 manufactured by MICROCHIP.

To enable a clinician to identify the location of the sampler 10, the printed-circuit board 38 also carries a marker 48, best seen in FIG. 5. A suitable marker 48 is a magnet, such as a neodymium magnet. Such a marker 48 can be tracked using a magnetometer.

In a preferred embodiment, the voltage source 40 maintains a 3.6 voltage and the first and second heaters 32, 34 comprise ten-ohm resistances. The resulting ohmic loss provides sufficient heat to raise the paraffin's temperature to a melting point of between 37° C. and 45° C. in under a minute. The amount of wax in the first and second plugs 28, 30 is optimized to achieve a desired melting time and re-solidification time.

In some embodiments, the sampler 10 includes a sensor that senses its local environment. The controller 36 controls operation of the first and second switches 42, 44 based on the output of the sensor. Accordingly, the sampler 10 is fully autonomous and requires no external intervention during sampling.

The illustrated sampler 10 has a length of between fifteen and thirty millimeters and a diameter of between five and fifteen millimeters. This permits sampling of between a hundred and three hundred microliters of fluid. A practical way of manufacturing such a sampler 10 is to print it using a biocompatible resin that is cured by exposure to ultraviolet radiation. A suitable resin is that which is ordinarily used to make dentures. An example of such a resin is Dental SG resin from FORMLABS.

The osmotic membrane 24 is manually inserted between the channel 18 and the sample chamber 22 and epoxied into place using a resin that is cured by exposure to ultraviolet radiation. A syringe injects a finely-powdered salt into the sample chamber 22. The resulting high salt concentration creates an osmotic pressure that drives a distal flow of sample from the inlet 16, through the channel 18, and into the sample chamber 22 during a sampling interval after between the melting of the first plug 28 and the re-solidification of the second plug 30.

The osmotic pressure results from the osmotic membrane's pore size, density, and thickness as well as the salt gradient across the osmotic membrane 24. A high osmotic pressure is useful for sampling more viscous gut fluids and to promote faster collection of gut fluid, thereby prompting spatial localization of the sample.

FIG. 10 shows an embodiment having plural channels 18, each having a corresponding inlet 16 and valve 20 as described in connection with FIGS. 5-9. Such an embodiment promotes reliability since there is a possibility that solid matter will clog a channel 18, thus rendering it inoperable.

In the embodiment of FIG. 10, the cap 12 comprises four inlets 16 that lead into a stilling basin 50 to which four channels 18 connect. Florescent markers 52 extend from the osmotic membrane 24 towards a distal end 15 of the body 14 to promote efficiency of recovery from fecal matter. A re-orientation magnet 54 provides a way to control the sampler's orientation from outside the patient. An exit nozzle 56 at the body's distal end 15 permits salt solution to exit the sample chamber 22 to make room for incoming sample arriving through the channels 18.

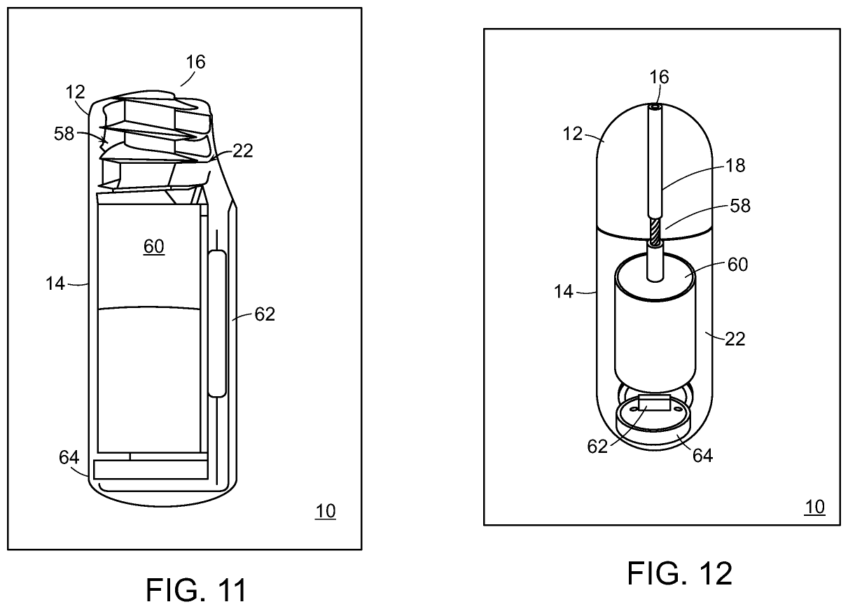

FIGS. 11 and 12 show embodiments in which active rather than passive sampling is carried out. Both embodiments feature a sampler 10 comprising a rotating screw 58 and a motor 60 to drive the screw 58. A switch 62 connects a battery 64 to the motor 60, thereby providing a way to control the screw's rotation. In a preferred embodiment, the switch 62 is a reed switch that opens and closes in response to a magnetic field. Accordingly, it is possible to turn the motor 60 on and off by applying a magnetic field from outside the patient.

In the embodiment shown in FIG. 11, the rotating screw 58 is large and hence the inlet 16 is also large. As a result, the embodiment shown in FIG. 11 samples large volumes relatively quickly. On the other hand, the large inlet 16 means that that the sample is prone to leaking out and also prone to being contaminated.

An alternative embodiment, shown in FIG. 12, the inlet 16 opens into a microfluidic channel 18. The screw 58 extends from the motor 60, through the microfluidic channel 18, and towards the inlet 16. As such, the inlet 16 is much smaller in aperture than that shown in the embodiment of FIG. 11. In such an embodiment, the channel 18 is sufficiently narrow so that a functioning screw 58 can be made by twisting two wires together to form a double-stranded helix.

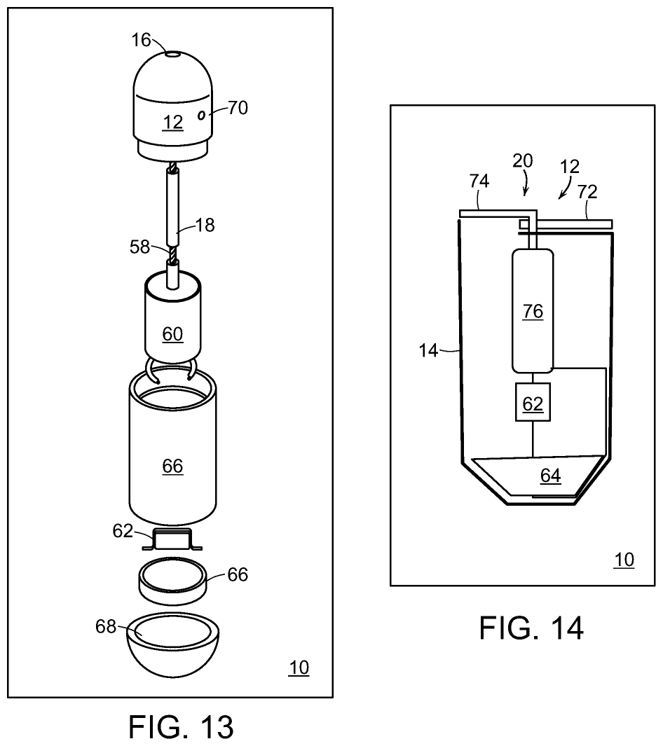

FIG. 13 shows an exploded view of the sampler 10 in FIG. 12 in which it the motor 60 is visible outside of its casing 66 and in which a distal cap 68 that holds the battery 64 can be seen. Additionally, FIG. 12 shows a vent 70 in the proximal cap 12 to permit air to escape as the supply chamber 22 fills with liquid sample.

In a preferred embodiment, the sampler 10 shown in FIGS. 12 and 13 is twenty-eight millimeters long and nine millimeters in diameter with a sample volume of about one hundred and fifty microliters to about two hundred microliters. The small inlet 16 reduces contamination. In addition, the sampler 10 is particularly suitable for sampling of viscous fluids.

In operation, the sampler 10 is coated with an enteric coating that dissolves in response to exposure to fluid having a pH corresponding to that of the region to be sampled. Once the enteric coating has been dissolved, the switch 62 transitions into a sampling state to begin the sampling process and then transitions into a non-sampling state to halt the sampling process.

In some embodiments, the switch 62 is made to transition between states in response to an externally applied signal, as has already been discussed in connection with FIGS. 5-9. In other embodiments, the sampler 10 comprises sensors that sense the local environment and that rely on those sensor signals to transition into a sampling state.

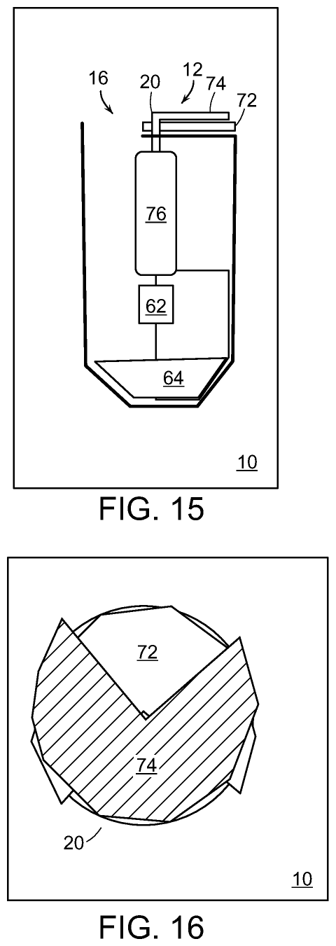

In another embodiment, shown in FIGS. 14 and 15, the proximal cap 12 comprises a valve 20 formed by a stationary panel 72 and a movable panel 74. A valve actuator 76 couples to the movable panel 74. In the embodiment shown, the actuator 76 is a motor that causes rotation. Embodiments include those in which the motor 60 is a stepper motor and those in which it is a de motor.

A switch 62 connects a battery 64 to the actuator 76, thereby causing the movable panel 74 to transition between a closed state, which is shown from the side in FIG. 14 and from the top in FIG. 16, and an open state, which is shown from the side in FIG. 15 and from the top in FIG. 17. In the embodiment shown in FIGS. 14-17, the transition between states arises as a result of swinging the movable panel around a pivot axis defined by the actuator 76.

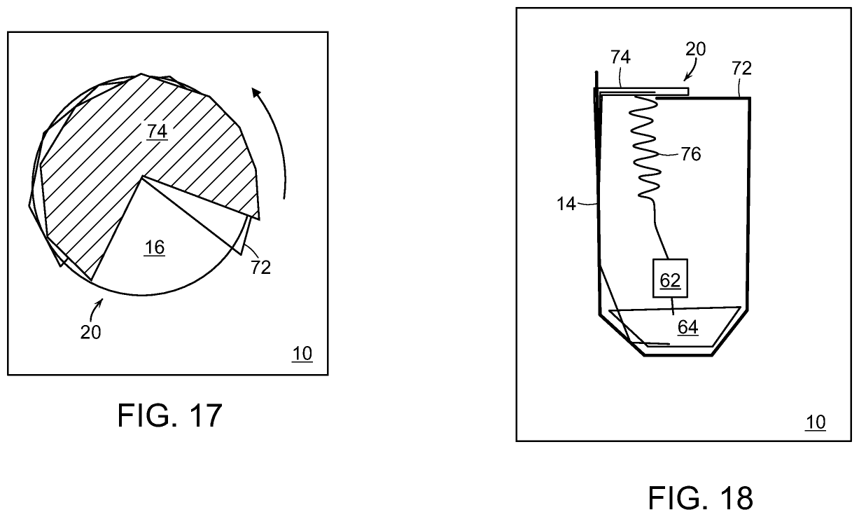

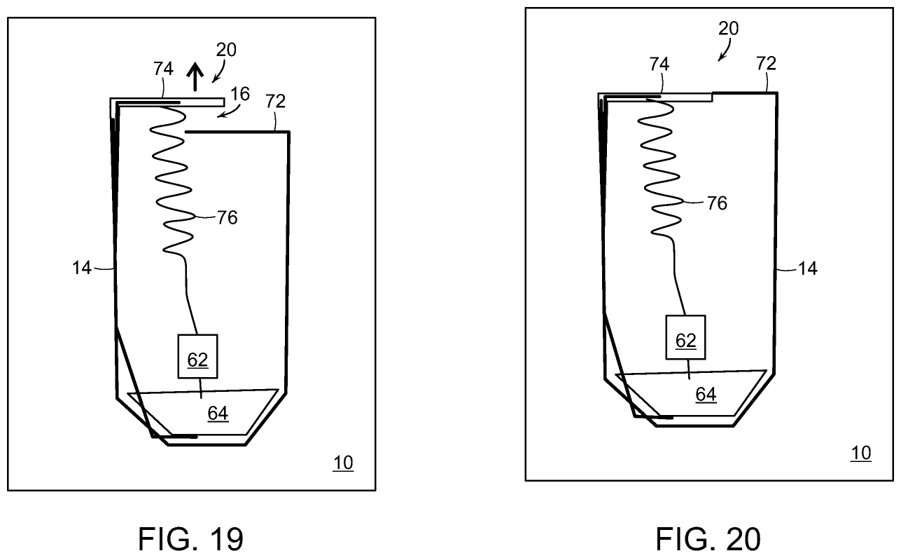

In an alternative embodiment, shown in FIGS. 18-19, the movable panel 74 is one that is raised and lowered. In this embodiment, the actuator 76 is implemented by a shape-memory material that transitions between an expanded state and a resting state in response to temperature. In such embodiments, the switch 62 causes flow of current, which results in ohmic heating that causes a transition in the shape-memory material's state.

In the embodiment shown in FIGS. 18-19, the movable panel 74 rests on stationary panel 72 when the actuator 76 is in its resting state. When the switch 62 permits current flow, the resulting heat causes the actuator 76 to transition into its expanded state, thus raising the movable panel 74 and forming an inlet 16 for collecting a sample.

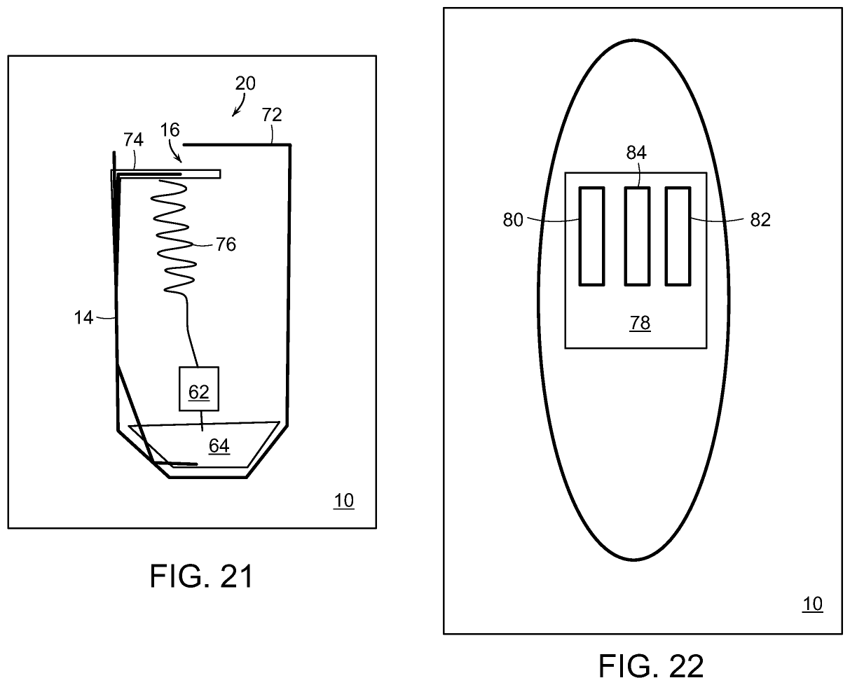

An alternative embodiment, shown in FIGS. 20-21 operates in a manner that is the converse to that shown in FIGS. 18-19. In the embodiment shown in FIGS. 20-21, the movable panel 74 lies below the stationary panel 72 when the actuator 76 is in its resting state. This forms an inlet 16 through which sampling takes place. Upon being heated, the actuator 76 transitions into its expanded state, thus raising the movable panel 74 so that it presses against the stationary panel 72 and closes the inlet 16.

In some embodiments, the switch 62 is a reed switch that can be controlled by an externally-generated magnetic field. In other embodiments, the switch 62 is coupled to a wireless transceiver, in which case an external transmitter controls the switch 62.

Some embodiments of the switch 62 include an antenna and a radio-frequency receiver, with or without a microcontroller that causes a transistor to transition between conducting and non-conducting states. In such cases, frequencies less than a gigahertz are preferable to avoid excessive losses when propagating through tissue.

Other embodiments of the switch 62 rely on acoustic energy to cause a transition between states. An example of such a switch 62 is one that uses a piezoelectric material that vibrates in response to incident acoustic energy and thus produces a voltage that is then rectified and used to control a transistor's state.

Still other embodiments of the switch 62 rely on light to cause a transition between states. An example of such a switch 62 is one that includes an optical receiver that is sensitive to infrared or near-infrared light.

Among the foregoing embodiments of the sampler 10 are those that require external intervention to begin the sampling process. These embodiments also exist in forms that avoid the need for such external intervention. Among these embodiments is that shown in FIG. 22, in which a sensor 78 senses a biomarker and, upon doing so, provides a signal to the controller 36. Preferably, the biomarker is one that changes as a function of location within the gut. Examples of such biomarkers include oxygen concentration and concentrations of electron donors or acceptors.

Embodiments that rely on spatially-variable biomarker levels make it possible to program the controller 36 to begin sampling at a particular region within the gut. For example, if a sample from the intestine is sought, a sensor 78 that senses a high concentration of electron acceptors provides a basis for inferring that the sampler 10 is still traversing the stomach whereas a drop in electron-acceptor concentrations would imply that the sampler 10 has left the stomach and begun its journey through the intestine.

Among these are embodiments in which the controller 36 autonomously halts sampling. Such embodiments include those that halt sampling in response to another signal from the sensor 78, in response to the lapse of some predetermined time, or in response to the intake of some predetermined volume of the sample.

In some embodiments, the sensor 78 is implemented as a potentiometric sensor that is suitable for measurement of concentrations of electron donors and/or electron acceptors. In other embodiments, the sensor is amperometric. Such sensors are useful for measurement of oxygen concentration.

In other embodiments, the sensor 78 measures a vector of biomarkers. An example of such an embodiment is one that measures a vector that includes a component indicative of dissolved oxygen concentration and a component indicative of electron-acceptor concentration or electron-donor concentration. Additional biomarkers include short-chain fatty acids, acetate, propionate, lactate, and bile acids. Other examples of biomarkers include constituents of colonic flatus, including methane.

FIG. 22 shows an example of a sensor 78 that measures a vector of biomarkers. The sensor 78 comprises first and second working electrodes 80, 82 and a reference electrode 84. The electrodes 80, 82, 84 are screen-printed onto the surface of the sampler 10. The first working electrode 80 is made from carbon or carbon nanotubes with polyaniline and is useful for sensing electron-acceptor concentrations. The second working electrode 82 is made of silver, gold, or zinc and is useful for Clark-based oxygen sensing. The reference electrode is screen printed from a silver or silver chloride paste. Based on outputs of the electrodes 80, 82, 84, the controller 36 determines whether to begin sampling.

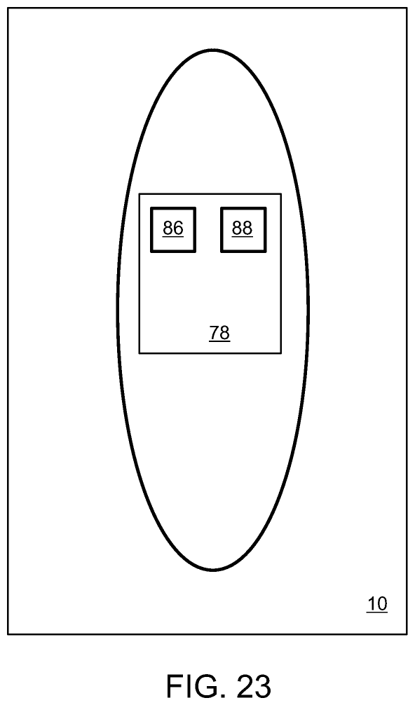

FIG. 23 shows another embodiment in which the sensor 78 comprises a first dye source 86 that releases a first dye and a second dye source 88 that releases a second dye. The first dye has properties that depend on electron-acceptor concentrations. Examples of a suitable first dye include Nile red, methyl red, and bromocresol purple. The second dye has properties that depend on dissolved oxygen concentration. Examples of a suitable second dye include a fluorescent dye that fluoresces in response to exposure to dissolved oxygen and a dye having a color that changes in response to exposure to dissolved oxygen.

In some embodiments, the dye is replaced by a chromophore that is sensitive to a particular biomarker, which can then be sensed by observing the chromophore. Examples of such biomarkers include short-chain fatty acids, acetate, propionate, lactate, and bile acids. Other examples of biomarkers include constituents of colonic flatus, including methane.

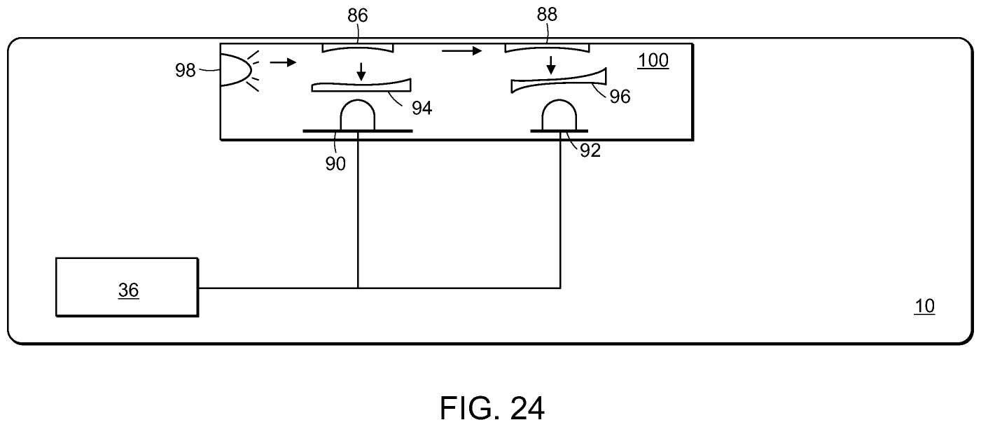

FIG. 24 shows components for inspecting the state of the dyes released by the first and second dye sources 86, 88 into the sample. These components include first and second photodetectors 90, 92 behind corresponding first and second filters 94, %. A light source 98 illuminates a sample-filled space 100 above the filters 94, %. In the illustrated embodiment, the first and second photodetectors 90, 92 thus provide time-varying signals indicative of the interaction of the respective first and second dyes with light from the light source 98.

Having described the invention, and a preferred embodiment thereof, what is new and secured by Letters Patent is:

1. An apparatus comprising an orally-administered gut sampler that traverses said gut and that obtains a sample of a bacterial population that is present at a selected location within said gut, said sampler comprising a body that extends from a proximal end to a distal end along a longitudinal axis thereof, wherein said body comprises an inlet and a sample chamber, wherein a sample of bacteria begins to pass into said inlet and into said sample chamber when said sampler arrives at said selected location.

2. The apparatus of claim 1, wherein said gut sampler further comprises a channel that extends from said inlet to said sample chamber, wherein said sample chamber comprises a bag having a volume that has been reduced as a result of having been folded within said body.

3. The apparatus of claim 1, wherein said gut sampler further comprises a channel and a valve, wherein said channel extends between said inlet and said sample chamber, wherein said valve is biased to block said channel, and wherein said sample chamber is configured to expand in volume, thereby causing a pressure differential across said valve, said pressure differential being sufficient to open said valve and diminishing over time such that said valve closes when said pressure differential is no longer sufficient to overcome said bias.

4. The apparatus of claim 1, wherein said gut sampler further comprises a coating around said body, wherein said coating is configured to begin to dissolve in response to exposure to fluid that is found at said selected location.

5. The apparatus of claim 1, wherein said gut sampler further comprises a channel and an osmotic membrane, wherein said channel extends distally from said inlet to said osmotic membrane, and wherein said sample chamber is distal to said osmotic membrane.

6. The apparatus of claim 1, wherein said gut sampler further comprises first and second pieces of a material and a channel, wherein said channel connects to said inlet, wherein said first piece forms a first plug that blocks said channel and second piece located so as to be transformable into a second plug that blocks said channel.

7. The apparatus of claim 1, wherein said gut sampler further comprises a valve and a pair of electric heaters that are disposed to heat first and second portions of said valve.

8. The apparatus of claim 1, wherein said gut sampler further comprises a magnet.

9. The apparatus of claim 1, further wherein said gut sampler further comprises a valve and a controller configured to control actuation of said valve.

10. The apparatus of claim 1, further wherein said gut sampler further comprises a voltage source, switches, and resistances, wherein said switches transition into a closed state that permits current driven by said voltage source to flow through said resistances.

11. The apparatus of claim 1, further wherein said gut sampler further comprises a fluorescent marker.

12. The apparatus of claim 1, wherein said gut sampler further comprises a plurality of inlets, of which said inlet is a first inlet, a stilling basin into which said inlets open, an osmotic membrane, and a plurality of channels, wherein each of said channels extends distally from said stilling basin to said osmotic membrane, and wherein said sample chamber is distal to said osmotic membrane.

13. The apparatus of claim 1, wherein said gut sampler comprises a screw and a motor coupled to said screw to cause rotation of said screw.

14. The apparatus of claim 1, wherein said gut sampler comprises a screw, a motor, and a tube, wherein said screw extends from said inlet to said motor, wherein said tube extends from said inlet to a point proximal to said motor where said tube is in fluid communication with said sample chamber, wherein said screw passes through said tube, wherein said motor rotates said screw about said longitudinal axis, and wherein said screw, when being rotated by said motor, draws sample through said tube and into said sample chamber.

15. The apparatus of claim 1, wherein said gut sampler comprises a panel that rotates about said longitudinal axis between first and second positions, wherein, in said first position, said panel opens said inlet, and wherein, in said second position, said panel closes said inlet.

16. The apparatus of claim 1, wherein said gut sampler comprises a panel that translates along said longitudinal axis between first and second positions that are offset from each other along said longitudinal axis, wherein, in said first position, said panel closes said inlet, and wherein, in said second position, said panel opens said inlet.

17. The apparatus of claim 1, wherein said gut sampler comprises a shape-memory material and a heater that heats said shape-memory material to cause a transition between first and second states of said shape-memory material, and wherein said transition between said first and second states of said shape-memory material causes said inlet to transition between being closed and being opened.

18. The apparatus of claim 1, wherein said gut sampler comprises a valve actuator that is actuated in response to a measurement of a property of an environment in which said gut sampler is located.

19. The apparatus of claim 1, wherein said gut sampler comprises electrodes and a controller that receives signals from said electrodes, wherein said electrodes are exposed to an environment in which said gut sampler is located, and wherein said controller opens and closes said inlet in response to said signals.

20. The apparatus of claim 1, wherein said gut sampler comprises a photodetector, a light source, a dye source, and a controller that receives a signal from said photodetector, wherein said light source is configured to illuminate a dye released by said dye source and said photodetector is disposed to view said dye and to provide a signal indicative of a state of said dye.
